(19) United States
(12) Patent Application Publication
(10) Pub. No.: US 2023/0143097 A1
(43) Pub. Date: May 11, 2023

(54) Compositions And Methods To Reduce And/Or Dissolve Calcium Deposits In Arterial Walls

(71) Applicant: Michael Woods, Keller, TX (US)

(72) Inventor: Michael Woods, Keller, TX (US)

(21) Appl. No.: 17/982,726

(22) Filed: Nov. 8, 2022

Related U.S. Application Data
(60) Provisional application No. 63/277,142, filed on Nov. 8, 2021.

(51) Int. Cl. A61K 38/48; A61K 9/00; A61K 31/593; A61K 31/122; A61P 9/10

(52) U.S. Cl. CPC A61K 38/4886; A61K 9/0053; A61K 38/482; A61K 31/593; A61K 31/122; A61P 9/10

(57) ABSTRACT

The invention includes pharmaceutical compositions that diminish and/or dissolve calcium deposits in arterial walls, methods for diminishing and/or dissolving calcium deposits in arterial walls, and methods for making and administering such compositions. The compositions comprise oral dosage forms of serrapeptase, nattokinase, vitamin D3 and vitamin K2 administered twice daily.

### CROSS REFERENCE TO RELATED APPLICATIONS

This application claims the benefit of U.S. Provisional Application Ser. No. 63/277,142, filed on Nov. 8, 2021, which is hereby incorporated by reference herein.

### FIELD OF THE INVENTION

The invention relates to compositions that diminish and/or dissolve calcium deposits in arterial walls, methods for diminishing and/or dissolving calcium deposits in arterial walls, and methods for making and administering such compositions.

### BACKGROUND OF THE INVENTION

Calcium deposits in arteries, such as coronary arteries, can cause serious medical complications. Methods to dissolve or otherwise remove such deposits have been attempted but suffer from various deficiencies. Such methods include surgical interventions, including inserting stents into vessels or replacing arteries with bypass surgery. These methods have several drawbacks and their effects are often transitory. Currently there is no methodology available to naturally dissolve calcium deposits in arterial walls. Better methods are needed.

### SUMMARY OF THE INVENTION

Embodiments of this invention comprise administering four agents that include serrapeptase, nattokinase, vitamin D3 and vitamin K2. The serrapeptase and nattokinase are administered in an oral dosage form, preferably twice a day on an empty stomach. The vitamin D3 and vitamin K2 are administered in oral dosage form, preferably twice a day with food.

The combination of these four agents can diminish and/or dissolve calcium deposits in arteries, including coronary arteries, as shown, for example, by coronary artery scans.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### DETAILED DESCRIPTION OF THE INVENTION

The invention provides a method for dissolving (e.g., treating, inhibiting, reducing, reversing, diminish and/or eliminating) calcium deposits in arteries. The method comprises the administration of four orally administered agents, preferably each agent two times per day. The four orally administered agents are serrapeptase, nattokinase, vitamin D3 and vitamin K2. The serrapeptase and nattokinase are preferably administered without food on an empty stomach. The vitamin D3 and vitamin K2 are preferably administered with food.

Serrapeptase is an enzyme that is sold as a dietary supplement. It can be produced by extraction from Serratia marcescens ATCC27117 strains. In certain embodiments the oral dosage is from about 1 to about 1000 mg. In preferred embodiments the oral dosage is about 7.5 mg to about 30 mg. In certain embodiments, the oral dosage is taken twice per day. In the most preferred embodiments, about 15 to about 60 mg is taken per day by administration of two oral dosages of about 7.5 mg to about 30 mg each and each time on an empty stomach without food (e.g., at least two hours after a meal). One of skill in the art can determine that other amounts of serrapeptase can be administered depending on the age, weight, sex, medical condition and other factors effecting or otherwise attributed to the patient.

Nattokinase is an enzyme that is sold as a dietary supplement. It can be produced by extraction from nattō or produced by recombinant means and in batch culture. In certain embodiments the oral dosage is from about 1 to about 1000 mg. In preferred embodiments the oral dosage is about 100 mg. In certain embodiments, the oral dosage is taken twice per day. In the most preferred embodiments, about 200 mg is taken per day by administration of two oral dosages of about 100 mg each and each time on an empty stomach without food. One of skill in the art can determine that other amounts of nattokinase can be administered depending on the age, weight, sex, medical condition and other factors effecting or otherwise attributed to the patient.

Vitamin D3 is also known as cholecalciferol and it is sold as a dietary supplement. In certain embodiments the oral dosage is from about 1 mcg to about 250 mcg per dose, twice per day, and in more preferred embodiments from about 75 to about 150 mcg, twice per day. In the most preferred embodiments the oral dosage is about 125 mcg (5000 international units). In certain embodiments, the oral dosage is taken twice per day. In certain embodiments, about 250 mcg is taken per day by administration of two oral dosages of about 125 mcg each and each time with food. One of skill in the art can determine that other amounts of vitamin D3 can be administered depending on the age, weight, sex, medical condition and other factors effecting or otherwise attributed to the patient.

Vitamin K2 is also known as menaquinone and it is sold as a dietary supplement. A preferred form of vitamin K2 is MK-7. In certain embodiments the oral dosage is from about 1 mcg to about 250 mcg per dose, twice per day, and in more preferred embodiments from about 50 mcg to about 150 mcg per dose, twice per day. In the most preferred embodiments the oral dosage is about 200 mcg per day. In certain embodiments, the oral dosage is taken twice per day.

In the most preferred embodiments, about 200 mcg is taken per day by administration of two oral dosages of about 100 mcg each and each time with food. One of skill in the art can determine that other amounts of vitamin K2 can be administered depending on the age, weight, sex, medical condition and other factors effecting or otherwise attributed to the patient.

In certain preferred embodiments, the serrapeptase is provided in an oral dose of between about 7.5 to about 30 mg, twice per day; the nattokinase is provided in an oral dose of between about 50 mg and 150 mg, twice per day; the vitamin D3 is provided in an oral dose of between about 75 mcg and 175 mcg, twice per day; and the vitamin K2 is provided in a dose of between about 50 mcg and about 150 mcg, twice per day.

In certain embodiments the combination of at least the four agents (viz., serrapeptase, nattokinase, vitamin D3 and vitamin K2) is taken twice per day for at least one day, more preferably for six months, and most preferably for one year. In other embodiments the combination is taken twice per day for two or more years (or shorter) until a noticeable improvement is obtained in the health of a patient's blood vessels, such as diminished calcium deposits in arteries such as coronary arteries. One of skill in the art can determine that the combination can be taken for different amounts of time depending on the age, weight, sex, medical condition and other factors effecting or otherwise attributed to the patient.

As used herein, in certain embodiments an “effective amount” is an amount that results in the desired effect as compared to a control—an amount that treats, inhibits, reduces, reverses, diminishes and/or eliminates calcium deposits on arteries when used with the other three agents.

As used herein, in certain embodiments a “patient” means an animal and in preferred embodiments a “patient” includes human beings.

The compositions disclosed herein may be orally administered, for example, with an inert diluent or with an assimilable edible carrier, or they may be enclosed in an oral dosage form such as a hard or soft shell gelatin capsule, or they may be compressed into tablets. For oral therapeutic administration, the active compounds may be incorporated with excipients and used in the form of ingestible tablets, buccal tablets, chewable tablets, disintegrating (e.g., Quick-Melt) tablets, troches, capsules, gummies, elixirs, suspensions, syrups, wafers, and the like. In preferred embodiments, such compositions and preparations should contain at least 0.1% of active compound. The percentage of the compositions and preparations may, of course, be varied and may conveniently be between about 1 to about 60% of the weight of the unit. The amount of active compounds in such therapeutically useful compositions is such that a suitable dosage will be obtained.

In certain preferred embodiments, the serrapeptase and nattokinase are combined in one dosage form. In other preferred embodiments, the vitamin D3 and vitamin K2 are combined in one dosage form. In certain other preferred embodiments, all four of the agents (viz., serrapeptase, nattokinase, vitamin D3 and vitamin K2) are combined in one dosage form that may or may not comprise excipients that provide at least some control of or preference for the release of the agents into the same or different parts of the digestive system.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, each of the four agents, or pharmaceutically acceptable salts thereof, may be incorporated into sustained-release preparations and formulations.

Without being construed to be a limitation of this invention, a theory of the invention is that the serrapeptase and nattokinase may dissolve fibrin accumulation in calcium deposits in the arteries. Serrapeptase and nattokinase also exhibit strong anti-inflammatory effects in the body which may help mitigate one of the core conditions that lead to calcium accumulation. The vitamin D3 may aid the body in properly absorbing calcium. The vitamin K2 may aid the body in having calcium delivered to the bones rather than to accumulate in soft tissue. When administered together the four agents act additively, or in preferred embodiments, more than additively, to dissolve calcium deposits. However, this theory of the invention and/or proposed mechanism of action is offered as background as potential effects and should not be construed to limit how the invention works or limit the invention to a particular mechanism of action, and the four oral agents may have additional and/or different effects than these and still be included within this invention.

### Example 1

In this prospective example, a patient with calcium deposits on their coronary arteries as shown by a coronary artery scan is administered effective amounts of oral doses of serrapeptase (20 mg) and nattokinase (100 mg) twice a day, each time on an empty stomach without food. The patient is also administered effective amounts of oral doses of vitamin D3 (125 mcg) and vitamin K2 (100 mcg of MK-7) twice a day, each time with food (e.g., with a meal such as breakfast and dinner).

Another coronary artery scan of the patient is then performed after six months or longer of treatment. The patient's calcium deposits on their coronary arteries will be shown to have been diminished.

In this example, a method of dissolving calcium deposits in arteries is provided. The method comprises (a) administering an effective amount of an oral dosage form of serrapeptase twice a day, each time on an empty stomach and without food; (b) administering an effective amount of an oral dosage form of nattokinase twice a day, each time on an empty stomach and without food; (c) administering an effective amount of an oral dosage form of vitamin D3 twice a day, each time with food; and (d) administering an effective amount of an oral dosage form of vitamin K2 twice a day, each time with food. In this example, the serrapeptase is provided in an oral dose of between about 1 mg and about 1000 mg and more preferably between about 7.5 to about 30 mg; the nattokinase is provided in an oral dose of between about 1 mg and about 1000 mg and more preferably about 100 mg; the vitamin D3 is provided in an oral dose of between about 1 mcg to about 150 mcg and more preferably about 125 mcg; and the vitamin K2 is provided in a dose of between about 1 mcg and about 250 mcg and more preferably about 100 mcg.

### Example 3

In this example, a method of treating coronary artery disease in a patient is provided.

The method comprises: (a) administering an effective amount of an oral dosage form of serrapeptase twice a day, each time on an empty stomach and without food; (b) administering an effective amount of an oral dosage form of nattokinase twice a day, each time on an empty stomach and without food; (c) administering an effective amount of an oral dosage form of vitamin D3 twice a day, each time with food; and (d) administering an effective amount of an oral dosage form of vitamin K2 twice a day, each time with food. In this example, the serrapeptase is provided in an oral dose of between about 1 mg and about 1000 mg and more preferably between about 7.5 to about 30 mg; the nattokinase is provided in an oral dose of between about 1 mg and about 1000 mg and more preferably about 100 mg; the vitamin D3 is provided in an oral dose of between about 1 mcg to about 150 mcg and more preferably about 125 mcg; and the vitamin K2 is provided in a dose of between about 1 mcg and about 250 mcg and more preferably about 100 mcg.

In this example, a kit for treating calcium deposits in arteries is provided. The kit comprises an effective amount of oral dosage forms of serrapeptase, nattokinase, vitamin D3 and vitamin K2. In another kit of this invention, the kit also includes instructions for a patient to take (or be administered) oral dosage forms of (a) the serrapeptase and nattokinase twice a day on an empty stomach, and (b) the vitamin D3 and vitamin K2 twice a day with food. In this example, the serrapeptase is provided in an oral dose of between about 1 mg and about 1000 mg and more preferably between about 7.5 to about 30 mg; the nattokinase is provided in an oral dose of between about 1 mg and about 1000 mg and more preferably about 100 mg; the vitamin D3 is provided in an oral dose of between about 1 mcg to about 150 mcg and more preferably about 125 mcg; and the vitamin K2 is provided in a dose of between about 1 mcg and about 250 mcg and more preferably about 100 mcg.

### Example 5

In this example, a combination is provided that comprises effective amounts of oral dosage forms of serrapeptase, nattokinase, vitamin D3 and vitamin K2. In another example, the combination comprises oral dosage forms that are each selected from the group consisting of ingestible tablets, buccal tablets, troches, capsules, gummies, elixirs, suspensions, syrups or wafers. In still another example, in the combination, the serrapeptase and nattokinase are combined in one dosage form. In still another example, in the combination, the vitamin D3 and vitamin K2 are combined in one dosage form. In still another example, in the combination, the serrapeptase, nattokinase, vitamin D3, and vitamin K2 are combined in one dosage form with one or more additional excipients that provide for at least some control of the release of the serrapeptase, nattokinase, vitamin D3 and vitamin K2 into at least one of two different parts of the digestive system. In this example, the serrapeptase is provided in an oral dose of between about 1 mg and about 1000 mg and more preferably between about 7.5 to about 30 mg; the nattokinase is provided in an oral dose of between about 1 mg and about 1000 mg and more preferably about 100 mg; the vitamin D3 is provided in an oral dose of between about 1 mcg to about 150 mcg and more preferably about 125 mcg; and the vitamin K2 is provided in a dose of between about 1 mcg and about 250 mcg and more preferably about 100 mcg.

### Other Embodiments

Although the present invention has been described with reference to teaching, examples and preferred embodiments, one skilled in the art can easily ascertain its essential characteristics, and without departing from the spirit and scope thereof can make various changes and modifications of the invention to adapt it to various usages and conditions. Those skilled in the art will recognize or be able to ascertain using no more than routine experimentation, many equivalents to the specific embodiments of the invention described herein. Such equivalents are encompassed by the scope of the present invention.

We claim:

1. A method of dissolving calcium deposits in arteries, the method comprising:
a. administering an effective amount of an oral dosage form of serrapeptase twice a day, each time on an empty stomach and without food;
b. administering an effective amount of an oral dosage form of nattokinase twice a day, each time on an empty stomach and without food;
c. administering an effective amount of an oral dosage form of vitamin D3 twice a day, each time with food; and
d. administering an effective amount of an oral dosage form of vitamin K2 twice a day, each time with food.

2. The method of claim 1 wherein the serrapeptase is provided in an oral dose of between about 7.5 to about 30 mg, administered twice a day; the nattokinase is provided in an oral dose of between about 50 mg and 150 mg, administered twice a day; the vitamin D3 is provided in an oral dose of between about 75 mcg and 175 mcg, administered twice a day; and the vitamin K2 is provided in a dose of between about 50 mcg and about 150 mcg, administered twice a day.

3. A method of treating coronary artery disease in a patient, the method comprising:
a. administering an effective amount of an oral dosage form of serrapeptase twice a day, each time on an empty stomach and without food;
b. administering an effective amount of an oral dosage form of nattokinase twice a day, each time on an empty stomach and without food;
c. administering an effective amount of an oral dosage form of vitamin D3 twice a day, each time with food; and
d. administering an effective amount of an oral dosage form of vitamin K2 twice a day, each time with food.

4. The method of claim 3 wherein the serrapeptase is provided in an oral dose of between about 7.5 to about 30 mg, administered twice a day; the nattokinase is provided in an oral dose of between about 50 mg and 150 mg, administered twice a day; the vitamin D3 is provided in an oral dose of between about 75 mcg and 175 mcg, administered twice a day; and the vitamin K2 is provided in a dose of between about 50 mcg and about 150 mcg, administered twice a day.

5. A kit for treating calcium deposits in arteries, the kit comprising effective amounts of oral dosage forms of serrapeptase, nattokinase, vitamin D3 and vitamin K2.

6. The kit of claim 5 further comprising instructions to take the oral dosage forms of (a) the serrapeptase and nattokinase twice a day on an empty stomach, and (b) the vitamin D3 and vitamin K2 twice a day with food.

7. The kit of claim 5 wherein the serrapeptase is provided in an oral dose of between about 7.5 to about 30 mg, administered twice a day; the nattokinase is provided in an oral dose of between about 50 mg and 150 mg, administered twice a day; the vitamin D3 is provided in an oral dose of between about 75 mcg and 175 mcg, administered twice a day; and the vitamin K2 is provided in a dose of between about 50 mcg and about 150 mcg, administered twice a day.

8. A combination comprising effective amounts of oral dosage forms of serrapeptase, nattokinase, vitamin D3 and vitamin K2.

9. The combination of claim 8 wherein the oral dosage forms are selected from the group consisting of ingestible tablets, buccal tablets, troches, capsules, gummies, elixirs, suspensions, syrups or wafers.

10. The combination of claim 8 wherein the serrapeptase and nattokinase are combined in one dosage form.

11. The combination of claim 8 wherein the vitamin D3 and vitamin K2 are combined in one dosage form.

12. The combination of claim 8 wherein the serrapeptase, nattokinase, vitamin D3, and vitamin K2 are combined in one dosage form with one or more additional excipients that provide for at least some control of the release of the serrapeptase, nattokinase, vitamin D3 and vitamin K2 into at least one of two different parts of the digestive system.

13. The combination of claim 8 wherein the serrapeptase is provided in an oral dose of between about 7.5 to about 30 mg, administered twice a day; the nattokinase is provided in an oral dose of between about 50 mg and 150 mg, administered twice a day; the vitamin D3 is provided in an oral dose of between about 75 mcg and 175 mcg, administered twice a day; and the vitamin K2 is provided in a dose of between about 50 mcg and about 150 mcg, administered twice a day.
